(19) United States
(12) Patent Application Publication
(10) Pub. No.: US 2009/0069739 A1
(43) Pub. Date: Mar. 12, 2009

(54) METHODOLOGY FOR NON-SURGICAL FACIAL SCULPTING AND LIFT

(76) Inventor: Ehab Mohamed, Beverly Hills, CA (US)

Correspondence Address: KAUTH , POMEROY , PECK & BAILEY ,LLP, P.O. BOX 19152, IRVINE, CA, 92623, US

(21) Appl. No.: 12/204,614

(22) Filed: Sep. 4, 2008

Related U.S. Application Data
(60) Provisional application No. 60/970,695, filed on Sep. 7, 2007.

(51) Int. Cl. A61N 1/00; A61B 18/00; A61B 19/00

(52) U.S. Cl. 604/20; 606/33; 128/898

(57) ABSTRACT

Non-surgical methods and systems of facial restoration, and more particularly a multi-technique method utilizing tissue tightening, collagen shortening and remodeling with long term fillers to provide for non-surgical facial sculpting and lift are provided. The methods and systems address the predictable pattern of multi-layered volume loss of tissue from the skin to the bone as a result of aging.

### CROSS REFERENCE TO RELATED APPLICATIONS

This application claims the benefit of U.S. Provisional Patent Application No. 60/970,695, filed on Sep. 7, 2007, the entire disclosure of which is hereby incorporated by reference as if set in full herein.

### FIELD OF THE INVENTION

The current invention is directed generally to non-surgical methods of facial restoration, and more particularly to a multi-technique method utilizing tissue tightening, collagen shortening and remodeling with long term fillers to provide for non-surgical facial sculpting and lift.

### BACKGROUND OF THE INVENTION

During the normal aging process there are predictable events that happen in the face. Indeed, the hallmarks of aging include sagging of the upper eye lids, drooping of the eye brows, eye bags, a generally tired look to the eyes, dark circles around the eyes or hollow areas around the eyes, drooping of the cheek and hollow or flat cheeks, deep lines around the mouth (nasolabial folds) and smile lines (marionette lines), and jowls with neck sagging. These aging hallmarks can be combined with recession (concavity) at the bridge of the nose, as well as flattening of the nasal profile, recession of the chin area, and thinning out and loss of volume in the lips.

Conventional thought has been that these effects are principally caused by the aggregate effects of gravity on less resilient aging skin. As a result, physicians of all types have adopted practices aimed specifically at redressing these gravity induced effects.

For example, plastic surgeons have for a long time addressed aging by lifting and tightening the skin, the fascia and muscle. To accomplish this, surgeons transpose or shorten these structures to allow higher placement and replacement of the different sagging hallmarks. However, this lifting often creates an unnatural (plastic surgery) look because it fails to address the concomitant loss of volume in the face.

A new and more recent trend is to transpose fat into the face to achieve some level of volume replacement. However, fat intake at the new site is unpredictable and fluctuates with weight. Also, exactly locating the replacement fat is difficult and cannot correct all these problems. Accordingly, even in an ideal world these surgical solutions will never restore the natural youthful look desired by patients because they lack correction of the precise transition areas and compartments of the face, they also fails to address the loss of bone mass, muscle mass, skin thickness and collagen

In contrast, dermatologists have devised a plethora of new lasers and fillers to resurface the skin and reduce fine lines and wrinkles. However, while these techniques correct problems with the surface of the skin, they lack significant volume replacement because they cannot address the lost volume in bone, muscle and fat. Moreover, current treatment regimes emphasize the placement of these fillers in the deep dermal layers and not subcutaneous replacement, again reducing the “naturalness” of the finished look of the patient.

Others prefer regional filler replacement; however, the placement site is usually too superficial to address the total volume replacement needed. Also, compartmental replacement or regional improvement ignores the essence of the normal continuum of the face and the beauty of a freely flowing contour. For example, getting rid of the lines around the mouth but ignoring the deep grove between the lower eye lids and the cheek, and/or leaving the cheeks in a lower position and flat results in a fuller lower face and can actually heighten the contrast of the lost volume of the mid-face (cheeks, tear trough and lower eye lids). This combination of factors can lead to a far less aesthetic result. The same can be said of the upper face (eye brows, upper eye lids and periorbital area).

Another technique co-developed by both dermatologists and plastic surgeons uses a variety of skin tightening devices that focus on the deep dermal collagen and, in some cases, the fibrous septae, which connects the dermis to the deep facial fascial layer. The best example is a monopolar radio-frequency device sold, for example, under the tradename Thermage™. Again, while such devices solve part of the problem by addressing the stretch of the fascial layer, and can even induce collagen remodeling, they still lack any significant volume replacement capability. For example, as some people age the lower orbital rim (concave area underneath the lower eye lid) becomes recessed. In others the muscle and tissue that was in the face sags from its position and forms deep lines around the mouth and cheeks. The drooping and sagging of the mid-face muscle just inferior to the lower eye lid causes a loss of volume support for the eyelid. This in turn causes the skin to stretch and sag with the formation of dark circles, eye bags and puffiness of the lower eye lids (eye bags). Laser resurfacing or surgery can address the sagging of skin and removal of the puffiness, but it does not address the original problem of mid-face muscle sagging and loss of support, which leads to recurrence, extortion (an eye lid that is turned inside out), or in some cases a worsening of the tear trough deformity and caving of the area underneath the eyes.

There have been some attempts to combine a variety of different technologies. However, none of these combined techniques use a coherent pattern that addresses all the layers of the face in a consistent fashion tailored to the patient's unique pattern of volume loss, wrinkle and sagging.

### SUMMARY OF INVENTION

The current invention is directed generally to non-surgical methods of facial restoration, and more particularly to a multi-technique method utilizing tissue tightening, collagen shortening and remodeling with long term fillers to provide for non-surgical facial sculpting and lift.

In one embodiment, tissue tightening is provided by defined vector facial layer skin contraction to predetermined regions of a face and injecting fillers to provide volume replacement from facial bone to facial skin.

In one embodiment, skin tightening along with a multi-layer total volume replacement using long-lasting fillers is provided. In another embodiment the skin tightening involves the use of monopolar radiofrequency treatments, such as, for example, Thermage®. In such an embodiment the total energy (number of passes and the energy of the radiofrequency used) applied to any particular area of the face is tailored for the particular tissue type in question.

In one embodiment, a facial restoration system includes a radio frequency generator, a monopolar electrode instrument releasably coupled to the radio frequency generator and provides defined vector facial layer skin contraction to predetermined regions of a face, and an injection system injecting fillers to provide volume replacement from facial bone to facial skin.

In still another embodiment, the fillers are applied in at least three different layers of the facial tissue, including the near bone, the muscle, and the subcutaneous. In such an embodiment, the type of filler and volume of filler used is dependent on the type of tissue into which the injection is placed.

In yet another embodiment, the technique also includes skin resurfacing techniques.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1 provides a flow chart of one exemplary methodology of the current invention;

FIG. 2 provides a schematic diagram of the tissue cross-section of a face overlaid with exemplary tissue tightening parameters in accordance with the current invention;

FIGS. 3a & 3b provide schematic diagrams of the tissue cross-sections of a face overlaid with exemplary injection parameters in accordance with the current invention;

FIGS. 4a & 4b provide schematic diagrams of the tissue cross-sections of a face overlaid with exemplary injection parameters in accordance with the current invention; and

FIG. 5 provides a block diagram of facial sculpting and lift system.

### DETAILED DESCRIPTION OF THE INVENTION

As described in brief above, the current invention is directed generally to non-surgical methods of facial restoration, and more particularly to a multi-technique method utilizing tissue tightening, collagen shortening and remodeling with long term fillers to provide for non-surgical facial sculpting and lift.

The current technique recognizes that the “hallmarks” of aging, including, for example, sagging of the upper eye lids, drooping of the eye brows, eye bags, a generally tired look to the eyes, dark circles around the eyes or hollow areas around the eyes, drooping of the cheek and hollow or flat cheeks, deep lines around the mouth (nasolabial folds) and smile lines (marionette lines), jowls with neck sagging, recession (concavity) at the bridge of the nose, as well as flattening of the nasal profile, recession of the chin area, and thinning out and loss of volume in the lips, are all different stages of same problem. Specifically, all these problems can be traced, not to the effects of gravity as believed by the plastic surgery community for decades or just loss of deep dermal collagen and elastin as dermatological convention still holds, but rather it is mainly due to a predictable pattern of multi-layered volume loss of tissue types ranging inward from the skin to the bone.

Volume loss can start as early as late teens to mid-twenties or as late as the early thirties as many people achieve their maximal facial growth. The rate of volume loss, degree of loss and location are complex and highly dependent on multiple factors including genetics, nutrition, anti-oxidants intake/free radical production and stress, all of which can affect the programmed cell death (apoptosis) that usually leads to such volume loss. In addition, fluctuations in weight as a result of dieting or pregnancy can also dramatically affect the rate of volume loss in any particular patient.

Despite these individual variances, the types of changes seen as a result of this volume loss can be broken into the following categories:
Facial bone resorption (flattening/recession), which results from increased bone loss relative to bone production followed later by different degrees of osteoporosis. This process usually starts in the mid-thirties.
Facial muscle volume loss, which results from muscle fiber loss of about 2% for every year after a person reaches the age of 40.
Loss and degradation of the collagen layers of the deep fascial support (the strong collagen tissue sheets that extend around the muscle and have been described as attaching to bone anchor points to hold fascial compartments together in more or less firm or stable pattern). This loss of collagen causes distortion of the compartments of the face and a predisposition for displacement of the various compartments. These effects can be further exaggerated by volume loss, which allows the natural forces of gravity to exert more downward displacement on the smaller volumes that remain.
Subcutaneous fat loss in different regions of the face, which is highly variable from once person to another, but which constitutes one of the most important elements of tissue volume loss in the face. For example, it is believed that subcutaneous fat volume loss accounts for between 50 to 80% of total volume loss. Indeed, a recent research has debunked the long held belief that facial sagging happens due to the effects of gravity, instead implicating regional (compartmental) fat loss in the subcutaneous areas of the face that either causes the skin to sag, or at least makes it more prone to the effects of gravity. (Plastic and Reconstructive Surgery Journal, June 2007.)
Finally, the skin both dermal layer loss plenty of collagen with aging process, ground substance (hyaluronic acid), elastin as well as the thickness of the epidermal layer. This combined with sun damage make the skin less pliable, less elastic and prone to develop wrinkle both superficial and deep especially with some degree of loss of volume support.

The current invention uses a multi-layer filler injection technique that provides for the total volume replacement of lost facial tissue, in combination with defined vector fascial layer skin tightening to both recreate the lost volume from the skin layer to the bone layer and to achieve a smoothly contoured natural look. Using the techniques of the current invention results in the creation of facial contours that flow naturally from one area to another for all five essential layers of the facial structure to return a natural youthful look to the face.

Although general techniques will be discussed herein, one of ordinary skill in the art will understand that the precise injection volumes and fascial layer tightening regimes used must be customized and tailored to address the specific level and pattern of volume loss in each patient. However, the minor modifications to the technique required by the natural variations in the facial structure of each patient are well within the skill of the ordinary practioner when taken in combination with the supportive teachings of the current disclosure.

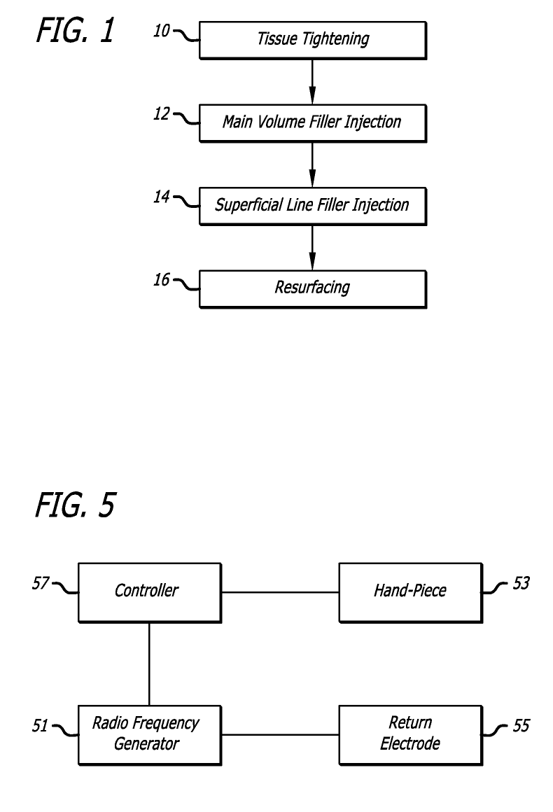

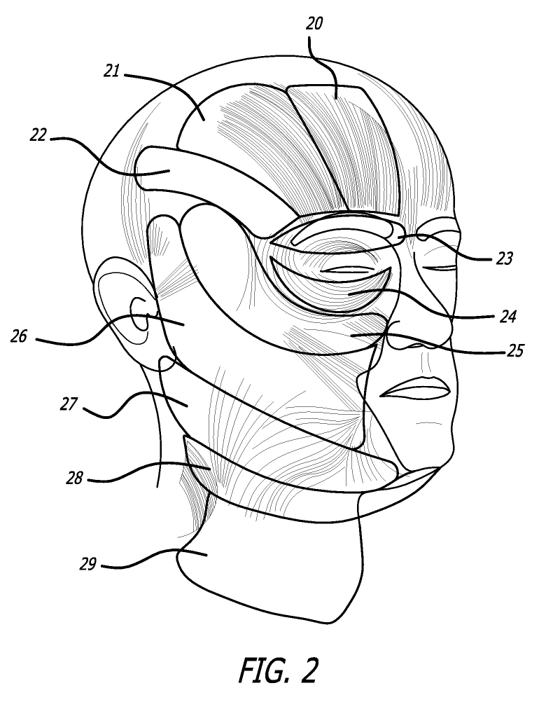

The details of the techniques of the current invention will be better understood with reference to FIGS. 2 to 4, which provide schematic diagrams of specific parameters for the injection and tissue tightening techniques. However, before turning to the details of the technique special reference is made to FIG. 1, which provides a flowchart of the various steps required by the multi-step process of the current invention.

As shown in FIG. 1, first tissue tightening is performed in a tailored fashion to provide defined vector fascial layer skin contraction to the different regions of the face (10). Tissue tightening is generally accomplished using a monopolar radiofrequency energy technique, which converts into heat in the deep collagen layers and fibrous septae, as well as fascia, and results in the immediate contraction of the supportive strong tissue. One exemplary monopolar radiofrequency tissue tightening technique is sold under the tradename Thermage®.

Tissue tightening is followed by the injection of fillers to provide entire volume replacement from bone to skin over the entire surface of the face (12). This process is preferably completed in a single step to allow for the best control over the overall contouring/sculpting of the face. In this step the filler(s) are injected in a controlled multi-path multi-layered and multi-directional pattern to provide volume replacement to the bone, muscle, subcutaneous fat and deep dermal layers of the face. In addition, if superficial lines exist, and these superficial lines or fine lines need correction a smoother filler may also be included at this stage of the process (14).

Finally in an optional step, resurfacing techniques can be performed if appropriate, such as, for example, if the patient does not have significant swelling (16). Such resurfacing can add to the synergistic effect of the upper/lower dermis collagen remodeling and fascial remodeling created by the tissue tightening and volume replacement steps discussed above.

The details of each of these steps are shown in FIGS. 2 to 4. As discussed above, in the first step of the current method the tissue is tightened using a monopolar radiofrequency energy technique. Unlike conventional techniques that teach tightening the tissue of the face in a uniform manner, the current technique is specifically directed to a technique that varies the number of passes and the amount of the energy used depending on the specific nature and location of the tissue in question. Each pass and degree of energy causes a certain percentage of the tissue in any region to contract. The results are both immediate (40% of final result) and delayed (60%) as a function of tissue healing response and new collagen remodeling. In general terms the current technique provides variable vector lift of different parts of the face by applying more passes and more energy (J/CM) in the direction of the desired lift. Although not to be bound by theory it is believed that this is because such a technique causes more collagen fiber contraction and more collagen production in the direction of the desired face lift.

FIG. 2 provides a schematic of the face overlaid with the ranges of energy and number of passes typically used in these areas. As shown, different treatment zones are defined to obtain differential vector lifts in specific areas, and are classified in this application as high/middle/low energy zones. The average energy used in each zone of radio-frequency is measured in Joules/cm² and varies from between 27 to 355 Joules/cm² depending on the type of tissue and desired lift. Also, the number of passes along same area is also provided and can vary from between 1 to a few dozen again depending on the type of tissue and desired lift. In short, the amount of tissue tightening, as measured by the contraction of collagen fibers and the production of new collagen, is in direct correlation with total energy received at that region or zone, which itself is the multiplication of the instantaneous energy (J/cm²) by the number of passes over same region. Higher energy levels cause a higher degree of shortening of collagen fibers. By controlling this shortening along uniquely placed strategic areas the tissue can be controllably lifted upward in a predictable dose response relationship to create a smooth aesthetic lift to the face. For example, the latter half of an eyebrow typically receives more passes and higher energy levels than other parts of the eyebrow to give a lateral lift to the brow, which is more aesthetically appealing than lifting the entire eye brow to the same degree.

Turning to the details of FIG. 2 and Table 1 below, in one particular embodiment, to provide the strongest lift to the cheek, as well as to decrease the sagging of eye bags and tear trough, the mid and lower face are divided in 3 parallel curved regions with the highest energy and number of passes of the monopolar radiofrequency in the top area of the desired areas to be lifted, followed by less energy and number of passes on the mid section, and the least energy and number of passes on the jowl area (lowest region). This energy distribution ensures the direction of tightening is upward and along the patient's natural bone attachment to the fascia. As shown in FIG. 2, the whole face and neck are done in a similar pattern. This will provide correction of the fascial support layers, the dermal collagen, and to some extent the fibrous septae in the subcutaneous layer.

TABLE 1
(FIG. 2)

| Region | Energy (J/cm²) | Passes | Ref. Numeral |
|---|---|---|---|
| Less Energy/Fewer Passes | 133 | 5 | 20 |
| High Energy/High Passes | 184 | 15 | 21 |
| Less Energy/Fewer Passes | 133 | 5 | 22 |
| High Energy/High Passes | 150 | 10 | 23 |
| High Energy/High Passes | 150 | 10 | 24 |
| High Energy/High Passes | 184 | 15 | 25 |
| Less Energy/Fewer Passes | 150 | 5 | 26 |
| Lowest Energy/Fewest Passes | 133 | 3 | 27 |
| High Energy/High Passes | 150 | 10 | 28 |
| Less Energy/Fewer Passes | 80 | 3 | 29 |

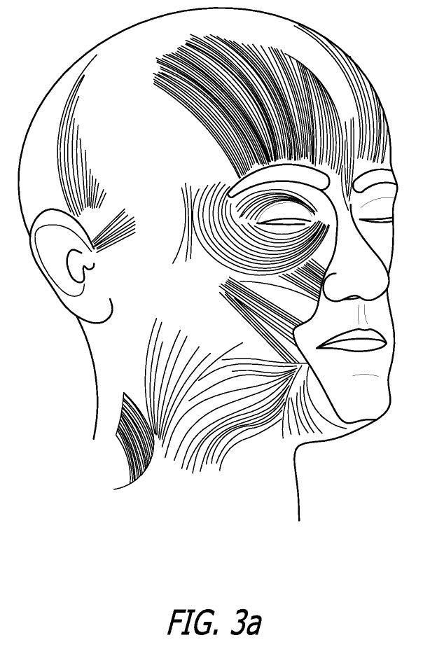

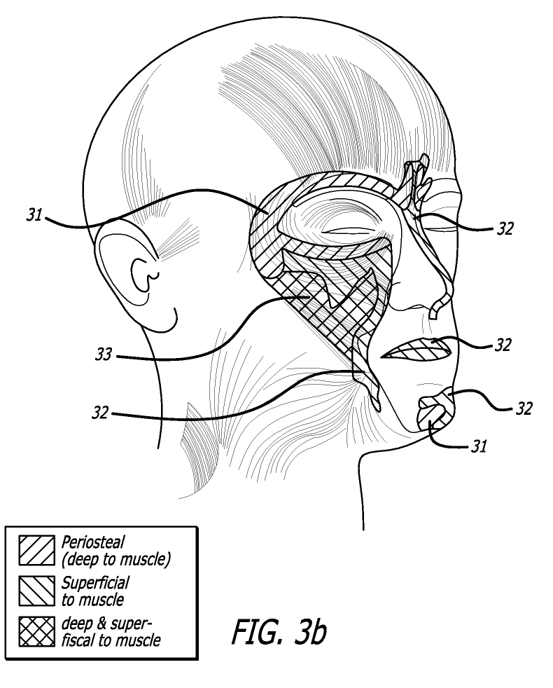

FIGS. 3 and 4 provide detailed schematic diagrams of some typical the filler injection profiles in accordance with the current invention. As discussed in the background, conventional filler injection techniques inject into the deep dermal only. Conventional dogma teaches that the injection of fillers into anything but these deep dermal regions, such as in subcutaneous tissue, provides no benefit because it will get absorbed quickly and won't produce collagen. There are two significant problems with these techniques. First, because of the fat content in the subcutaneous layer it surprisingly turns out to be the most important layer to replace. For example, it has now been determined that the subcutaneous layers of the face have fat bands that contain 10 times the collagen generating stem cells, and one of the advantages of new long terms fillers is that they can induce collagen production. Moreover, recent research has shown that there is a large fat component in this area where all wrinkles start. Also, most techniques instruct the use of a standard volume in all areas of the face. The current technique recognizes that these volume filling injections must be determined by the region of the face. If such differential injection is not used, the wrinkles in the face can actually be augmented by volume filling injection because of the over-expansion of certain areas of the face. This over-expansion also results in an imbalanced appearance in these areas.

In the current technique tissue tightening is followed immediately by the injection of long term fillers, such as those sold under the tradenames Radiesse™ and/or Artefill™ in all layers of the face. Specifically, the volume filling injections of the current technique are performed at four different layers depending on the region of the face. Specifically, the current technique calls for injections to be made in the deep layers such as the bone, muscle and subcutaneous tissue, as well as into the shallower deep dermal layer. The injections are made in a controlled multi-path, multi-layered and multi-directional pattern to create the major volume replacement in these layers, and to allow for the greatest consistency in volume replacement.

Diagrams showing the exact injection locations used in one exemplary embodiment or the current invention are shown in FIGS. 3 (oblique) and 4 (frontal) and further in tables 2-3. The areas shaded in black indicate submuscular (underneath the muscle—and periosteal) injections. The regions shaded grey are supra-muscular (superficial to muscle and injected in subcutaneous and deep dermal areas). The cross hatched areas indicate zones of the face in which both sub and supramuscular injections are made.

As will be understood, the actual volume of filler needed will depend on the level of correction needed. Although not to be bound by specific numbers, some exemplary volumes would be from around 7 ml in young patients less than 40 years to around 29 ml in patients above 60 years of age. The average amount of any injection would be on the order of 14 ml.

TABLE 2
(FIG. 3)

| Regions/Injections | Ref. Numerals |
|---|---|
| Periosteal (deep to muscle) | 31 |
| Deep and Superficial to Muscle | 33 (cross-hatched) |
| Superficial to Muscle | 32 |

TABLE 3
(FIG. 4)

| | Ref. | |
|---|---|---|
| Regions/Injections | Numerals | Description |
| Periosteal (deep to muscle) | 41a | Eye Brows - Deep to Muscle |
| | | (Periosteal) |
| Superficial to Muscle | 41b | Eye Brows - (Subcutaneous) |
| Periosteal (deep to muscle) | 42 | Periorbital to temporal - |
| | | Deep to Muscle |
| Periosteal (deep to muscle) | 43 | Cheek - Deep to Muscle |
| Superficial to Muscle | 44 | Lips - Superficial to Muscle |
| | | (Subcutaneous) |
| Periosteal (deep to muscle) | 45a, b | Chin - Deep to Muscle and |
| Superficial to Muscle | | Superficial to Muscle |
| Superficial to Muscle | 46 | Nasolabial folds - Superficial |
| | | to Muscle |
| Superficial to Muscle | 47 | Cheek - Deep to Muscle and |
| | | Superficial to Muscle |
| Periosteal (deep to muscle) | 48 | Tear through Deep to Muscle |
| | | and Superficial to Muscle |

Finally, if superficial lines exist and superficial lines or fine lines need correction a smoother filler can be used. Although any smoother filler may be used, preferred are those fillers known not to produce irregularities, such as, for example, fillers sold under the tradenames Perlane™, Restylane™, and Juvederm™.

Regardless of the actual volume of filler or type of filler used, the entire volume replacement from bone to skin including all areas should be done at one time to allow the highest level of control in contouring and sculpting the face. After injection, the fillers may be molded between the surgeon fingers to conform to the patient own natural bony structures to leave no irregularities. Such molding can be done up to 15-20 minutes following injection. The entire procedure should be done while the patient is sitting to appreciate the effects of gravity on the displacement of different compartments and hence the required volume correction.

In a final optional step, skin resurfacing can also be performed if the patient does not have significant swelling. Such resurfacing adds the further synergistic effect of upper lower dermis collagen remodeling to the fascial remodeling affected by the tissue tightening and long term volume filling injections of the previous steps.

Although specific tissue tightening and injection parameters are discussed above, it should be understood that the current invention is generally directed to any procedure that combines multilayered total volume replacement of all facial compartments with tissue tightening that is customized to achieve a specific vector face lift by applying differential energy levels to different areas of the face. The inventive technique of the current invention recognizes that enmeshing the two procedures together allows for far superior face lift and contouring by practioners.

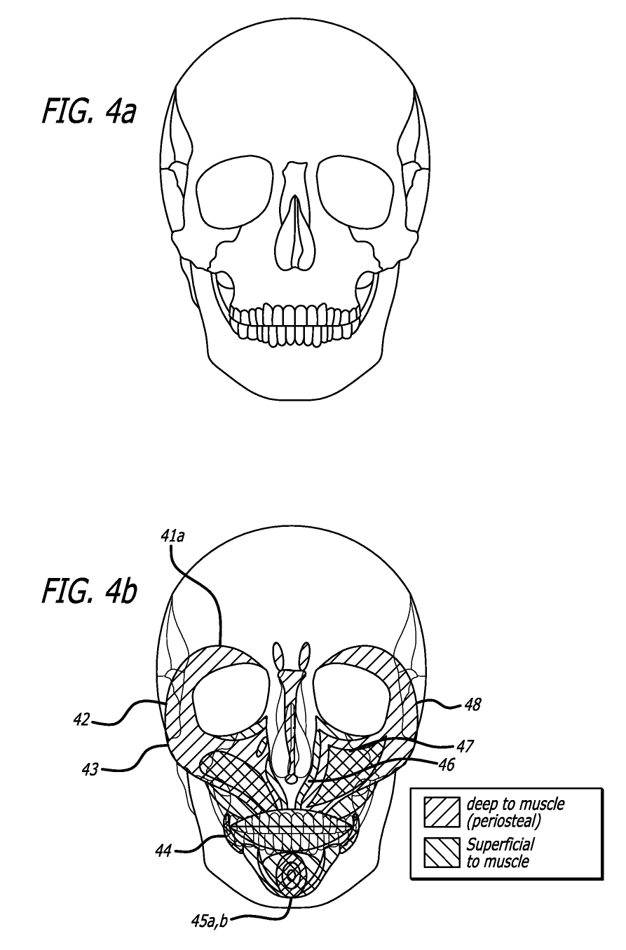

Referring now to FIG. 5, one exemplary system of providing the tissue tightening includes facial structuring medical instrument or hand-piece 53. In one embodiment, the medical instrument includes a monopolar high frequency hand-piece coupled to a radio frequency (RF) generator 51. The medical instrument also includes or is coupled with an injection system, e.g., one or more needles and with one more injection vials. The hand-piece applies RF energy to the patient's tissue and returns to a separate return electrode 55 in contact with the patient. Coupled or integrated into the RF generator or hand-piece is a controller 57 that monitors the cycles and use time of the RF energy being applied by the hand-piece. The controller in one embodiment includes an energy monitor configured to track and/or record the total RF energy applied by the hand-piece. In one embodiment, the energy monitor also records and/or tracks the number of passes and RF energy applied to predetermined regions of the patient's face. A mapping module integrated within or attached to the controller identifies a predetermined amount of passes and energy amount relative to the location of the hand-piece to the patient. In one embodiment, the mapping module includes a storage or database, removable, permanent or both, that stores records the predetermined regions of the face with a corresponding number of passes, amount of RF energy and/or total amount of RF energy to be applied to specific regions of the face. The location of the hand-piece can be identified and provided by the user or alternatively setup in a sequence of steps graphically displayed relative to a patient's body image or silhouette through a user interface coupled to the controller. Indicators such as visual, auditory and others can be provided to indicate the limits and/or indicate the completion of the current step and/or the initiation of the next step. For example, the hand-piece is moved to the next position and then activated thereby activating the next step. Indicators are also provided to indicate the number of passes and amount of energy through digital meter readouts or graphs displayed and/or printed out. Dials, knobs, buttons and other types of switches are also provided with the user interface to set and adjust the number of passes and/or amount of RF energy as desired by the user.

Similarly, in one embodiment, the injection system operates in a similar fashion, but, instead of RF energy, fillers are utilized. For example, the hand-piece includes or a separate hand-piece is utilized that has needles and containers holding the fillers to be used. When the injection system is included with the RF applicator of the hand-piece, the injection system is insulated to avoid shorts or other undesirable conductivity with the RF applicator. The injection system in one embodiment is retracted within the hand-piece when not in use and extended as it is used. Coupled or integrated into the injection system or hand-piece is a controller 57 that monitors the fillers being injected. In one embodiment, the controller also records and/or tracks the fillers injected to predetermined regions of the patient's face. A mapping module integrated within or attached to the controller identifies a predetermined amount of fillers relative to the location of the hand-piece to the patient. In one embodiment, the mapping module includes a storage media or database, removable, permanent or both, that stores records the predetermined regions of the face with a corresponding type and amount of filler to be injected to specific regions of the face. A user interface with indicators indicate the type and amount of filler being used through readouts or graphs displayed and/or printed out. Dials, knobs, buttons and other types of switches in one embodiment are also provided with the user interface to set and adjust or select the type and amount of filler as desired by the user. As such, the controller can indicate, monitor, facilitate and regulate the overall sequence of the process as shown in FIG. 2, for example, in addition to the specific application of RF energy and/or injection of fillers as specified throughout the description and as shown for example in FIGS. 3-4.

The whole concept of combining the process of tissue tightening, collagen shortening and remodeling with long term fillers that produce collagen in addition to total volume replacement, is to provide a synergistic and homogenizing effect on the face of patient that interweaves the new collagen production induced by the fillers with the collagen remodeling and tightening induced by a tightening technique like Thermage™. Such a combination of techniques enables a practioner to achieve significant tissue tightening and total volume replacement in a single session with significant improvement in the appearance of wrinkles and sagging. In addition, because of the differential application of these individual techniques the current procedure also produces more naturally flowing contours in the face. In short, the current non-surgical methodology results in the restoration of a youthful appearance with minimal recovery time that is far superior to any plastic surgery procedure or non-surgical dermal filler or tissue tightening procedure currently on the market.

Finally, it should be understood that while preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed is:

1. A method of performing facial restoration comprising:
tissue tightening by providing defined vector facial layer skin contraction to predetermined regions of a face; and
injecting fillers to provide volume replacement from facial bone to facial skin.

2. The method of claim 1 wherein the tissue tightening further comprises heating deep collagen layers and fibrous septae and fascia causing an immediate contraction of the supportive strong tissue.

3. The method of claim 1 wherein the injecting filler is performed immediately after the tissue tightening.

4. The method of claim 1 wherein the fillers are injected in a controlled multi-path multi-layered and multi-directional pattern.

5. The method of claim 1 further comprising injecting a smoother filler upon identification of superficial lines to be corrected.

6. The method of claim 1 further comprising resurfacing facial portions.

7. The method of claim 1 wherein the tissue tighten further comprises applying radio frequency energy to the predetermined regions of the face, the applied radio frequency energy used ranging from 27 to 355 Joules/cm².

8. The method of claim 7 wherein the applied radio frequency is applied through a number of passes ranging from 1 to 15.

9. The method of claim 8 wherein each pass and amount of energy causes a specific percentage of the predetermined region of the face to contract.

10. The method of claim 9 wherein about sixty percent of the predetermined region of the face contraction is delayed.

11. The method of claim 4 wherein the fillers injected include long term fillers injected at different layers in the face.

12. The method of claim 11 wherein the different layers in the face include near bone, muscle, subcutaneous and deep dermal layer.

13. The method of claim 4 wherein injecting fillers further comprises injecting two different fillers, a first filler being a sub-muscular injection and a second filler being a supra-muscular injection.

14. The method of claim 4 wherein a volume of injected filler ranges from 7 ml to 29 ml.

15. The method of claim 1 wherein the applied radio frequency energy is applied in an upward direction and along facial bone attachment to fascia.

16. A facial restoration system comprising:
a radio frequency generator;
a monopolar electrode instrument releasably coupled to the radio frequency generator and providing defined vector facial layer skin contraction to predetermined regions of a face; and
an injection system injecting fillers to provide volume replacement from facial bone to facial skin.

17. The system of claim 16 further comprising a controller monitoring an amount of radio frequency energy applied by the monopolar electrode instrument.

18. The system of claim 16 wherein the controller includes a mapping module and a map database, the map database having records a number of passes and radio frequency energy relative to a facial zone.

19. The system of claim 16 wherein the fillers are injected in a controlled multi-path multi-layered and multi-directional pattern, the fillers injected are one of a long term and smoother filler.

20. The system of claim 16 wherein the monopolar electrode instrument in contact with the face applies radio frequency energy to predetermined regions of the face, the applied radio frequency energy used ranges from 27 to 355 Joules/cm² and the applied radio frequency is applied through a number of passes ranging from 1 to 15.
